(19) United States
(12) Patent Application Publication
(10) Pub. No.: US 2025/0268767 A1
(43) Pub. Date: Aug. 28, 2025

(54) ELASTIC COVER FOR WEARABLE ARTICLE AND METHOD OF MAKING THEREOF

(71) Applicant: The Procter & Gamble Company, Cincinnati, OH (US)

(72) Inventors: Fengchun YE, Beijing (CN); Zhao ZHANG, Beijing (CN); Wei ZHOU, Guangzhou (CN); Jinrong FENG, Guangzhou (CN); Zhiqiang HUANG, Guangzhou (CN); Ruiming ZHOU, Guangzhou (CN)

(21) Appl. No.: 19/207,526

(22) Filed: May 14, 2025

Related U.S. Application Data
(63) Continuation of application No. PCT/CN2023/125442, filed on Oct. 19, 2023.

(30) Foreign Application Priority Data
Dec. 15, 2022 (WO) PCT/CN2022/139239

(51) Int. Cl. A61F 13/49; A61F 13/15

(52) U.S. Cl. CPC A61F 13/49019; A61F 13/15699; A61F 13/15707; A61F 13/15764; A61F 2013/15715; A61F 2013/1591

(57) ABSTRACT

Disclosed is an elastic cover comprising: an outer sheet, an inner sheet, a plurality of waist elastic bodies joined between the outer and inner sheets and extending transversely substantially parallel to each other, and a plurality of front leg elastic bodies joined between the outer and inner sheets and extending from the left front region through the crotch region to the right front region, a plurality of back leg elastic bodies joined between the outer and inner sheets and extending from the left back region through the crotch region to the right back region, wherein the front and back leg elastic bodies are joined to the outer and inner sheets by a leg elastic adhesive which is a hot melt adhesive applied in slot-die coating provided in a plurality of stripes intermittent in the longitudinal direction for joining the leg elastic bodies.

### CROSS REFERENCE TO RELATED APPLICATION

This application is a continuation of PCT Patent Application No. PCT/CN2023/125442, filed on Oct. 19, 2023, which claims priority, under 35 U.S.C. § 119, to PCT Patent Application No. PCT/CN2022/139239, filed Dec. 15, 2022, the entire disclosures of which are hereby incorporated by reference.

### FIELD

The present invention relates to an elastic cover for a wearable article, and method of making thereof.

### BACKGROUND

In recent years, various nonwoven materials have been suggested and used for making the garment-facing surface of the absorbent article to have certain functionality, such as softness, loftiness and breathability. Such nonwoven materials may be provided in relatively high caliper or high basis weight. In order to preserve the functionality of such materials, there is desire to prevent any region of the absorbent article from significantly stiffening due to manufacturing needs.

Absorbent articles such as diapers have areas of elasticity to provide a snug fit on the wearer. Such areas of elasticity are provided by elastic bodies to be used as waist bands and leg bands. In some configurations, it may be advantageous for certain areas of the elastic bodies to be free of elasticity. For example, it may not be desirous to have elasticity provided by leg elastic bodies in the crotch region (30) where an absorbent core overlaps. If elasticity is present in the absorbent core overlapping area, this may be uncomfortable for the wearer, and also cause unnecessary bunching up of material deteriorating the absorbing efficiency of the absorbent core. Removal of elasticity in such regions have commonly been conducted by bonding the leg elastic bodies to the supporting substrate by hot melt adhesive, and cutting them where hot melt adhesive is existing. In that the leg elastic bodies may extend over a certain area of the article, applying a high amount of hot melt adhesive over the entire lengths of the leg elastic bodies may lead to stiffening of the region. On the other hand, if not enough hot melt adhesive is applied over certain portions of the lengths of the leg elastic bodies, the leg elastic bodies may be dispositioned from the original position after loading of the absorbent core, which may lead to higher tendency of red marking and sagging.

Based on the foregoing, there remains a need for providing a reliable leg elastic body adhesion system which may have minimum influence on the planned position of the leg elastic bodies or the original desirable function of the material, which is cost efficient, and which has little influence on production speed.

### SUMMARY OF THE INVENTION

The present invention is directed to an elastic cover having a longitudinal direction, a transverse direction, a pair of side seams, and a pair of leg openings, wherein a pant is formed by joining the pair of side seams comprising:
a front region transversely extending between the pair of side seams;
a back region transversely extending between the pair of side seams; and
a crotch region longitudinally extending between the front and back regions;
the elastic cover comprising an elastic laminate comprising:
an outer sheet,
an inner sheet,
a plurality of waist elastic bodies joined between the outer sheet and the inner sheet and extending transversely substantially parallel to each other, and
a plurality of front leg elastic bodies joined between the outer sheet and the inner sheet and extending from the left front region through the crotch region to the right front region, a plurality of back leg elastic bodies joined between the outer sheet and the inner sheet and extending from the left back region through the crotch region to the right back region, wherein the front and back leg elastic bodies are joined to the outer sheet and inner sheet by a leg elastic adhesive which is a hot melt adhesive applied in slot-die coating;
wherein the leg elastic adhesive is provided in a plurality of stripes intermittent in the longitudinal direction for joining the leg elastic bodies, wherein each leg elastic adhesive stripe has a longitudinal dimension of no more than about 15 mm, and spaced apart from each other in the longitudinal direction with a spacing of no more than about 25 mm.

The present invention is also directed to a method of making the aforementioned elastic cover.

### BRIEF DESCRIPTION OF THE DRAWINGS

While the specification concludes with claims particularly pointing out and distinctly claiming the subject matter which is regarded as forming the present invention, it is believed that the invention will be better understood from the following description which is taken in conjunction with the accompanying drawings and which like designations are used to designate substantially identical elements, and in which:

FIG. 1 is a schematic perspective view of an embodiment of a wearable article of the present invention.

FIG. 2 is a schematic plan view of an embodiment of a wearable article of the present invention with the seams unjoined and removed, and in a flat uncontracted condition showing the wearer facing surface.

FIGS. 3A-3B are schematic plan views of embodiments of an elastic cover assembly of the present invention.

FIG. 3C is a schematic plan view of an elastic cover assembly of the prior art.

### DEFINITIONS

As used herein, the following terms shall have the meaning specified thereafter:

“Wearable article” refers to articles of wear which may be in the form of pants, taped diapers, incontinent briefs, feminine hygiene garments, and the like. The “wearable article” may be so configured to also absorb and contain various exudates such as urine, feces, and menses discharged from the body. The “wearable article” may serve as an elastic cover adaptable to be joined with a separable disposable absorbent insert for providing absorbent and containment function, such as those disclosed in PCT publication WO 2011/087503A.

“Pant” refers to disposable absorbent articles having a pre-formed waist and leg openings. A pant may be donned by inserting a wearer's legs into the leg openings and sliding the pant into position about the wearer's lower torso. Pants are also commonly referred to as “closed diapers”, “prefastened diapers”, “pull-on diapers”, “training pants” and “diaper-pants”.

“Longitudinal” refers to a direction running substantially perpendicular from a waist edge to an opposing waist edge of the article and generally parallel to the maximum linear dimension of the article.

“Transverse” refers to a direction perpendicular to the longitudinal direction.

“Proximal” and “distal” refer respectively to the position closer or farther relative to the longitudinal center of the article.

“Body-facing” and “garment-facing” refer respectively to the relative location of an element or a surface of an element or group of elements. “Body-facing” implies the element or surface is nearer to the wearer during wear than some other element or surface. “Garment-facing” implies the element or surface is more remote from the wearer during wear than some other element or surface (i.e., element or surface is proximate to the wearer's garments that may be worn over the disposable absorbent article).

“Disposed” refers to an element being located in a particular place or position.

“Joined” refers to configurations whereby an element is directly secured to another element by affixing the element directly to the other element and to configurations whereby an element is indirectly secured to another element by affixing the element to intermediate body(s) which in turn are affixed to the other element.

“Film” refers to a sheet-like material wherein the length and width of the material far exceed the thickness of the material. Typically, films have a thickness of about 0.5 mm or less.

“Water-permeable” and “water-impermeable” refer to the penetrability of materials in the context of the intended usage of disposable absorbent articles. Specifically, the term “water-permeable” refers to a layer or a layered structure having pores, openings, and/or interconnected void spaces that permit liquid water, urine, or synthetic urine to pass through its thickness in the absence of a forcing pressure. Conversely, the term “water-impermeable” refers to a layer or a layered structure through the thickness of which liquid water, urine, or synthetic urine cannot pass in the absence of a forcing pressure (aside from natural forces such as gravity). A layer or a layered structure that is water-impermeable according to this definition may be permeable to water vapor, i.e., may be “vapor-permeable”.

“Extendibility” and “extensible” mean that the width or length of the component in a relaxed state can be extended or increased.

“Elasticated” and “elasticized” mean that a component comprises at least a portion made of clastic material.

“Elongatable material”, “extensible material”, or “stretchable material” are used interchangeably and refer to a material that, upon application of a biasing force, can stretch to an elongated length of at least about 110% of its relaxed, original length (i.e. can stretch to 10 percent more than its original length), without rupture or breakage, and upon release of the applied force, shows little recovery, less than about 20% of its elongation without complete rupture or breakage as measured by EDANA method 20.2-89. In the event such an elongatable material recovers at least 40% of its elongation upon release of the applied force, the elongatable material will be considered to be “elastic” or “elastomeric.” For example, an elastic material that has an initial length of 100 mm can extend at least to 150 mm, and upon removal of the force retracts to a length of at least 130 mm (i.e., exhibiting a 40% recovery). In the event the material recovers less than 40% of its elongation upon release of the applied force, the elongatable material will be considered to be “substantially non-elastic” or “substantially non-elastomeric”. For example, an elongatable material that has an initial length of 100 mm can extend at least to 150 mm, and upon removal of the force retracts to a length of at least 145 mm (i.e., exhibiting a 10% recovery).

“Radial” means a direction running from the center of a drum toward a drum outer circumferential surface.

“Substrate” means a material which is primarily two-dimensional (i.e., in an XY plane) and whose thickness (in a Z direction) is relatively small (i.e., 1/10 or less) in comparison to its length (in an X direction) and width (in a Y direction). Non-limiting examples of substrates include a web, layer or layers or fibrous materials, nonwovens, films and foils such as polymeric films or metallic foils. These materials may be used alone or may comprise two or more layers laminated together. As such, a web is a substrate.

“Nonwoven” means a material made from continuous (long) filaments (fibers) and/or discontinuous (short) filaments (fibers) by processes such as spunbonding, meltblowing, carding, and the like. nonwovens do not have a woven or knitted filament pattern.

“Machine direction” means the direction of material flow through a process. In addition, relative placement and movement of material can be described as flowing in the machine direction through a process from upstream in the process to downstream in the process.

“Cross machine direction” means a direction that is generally perpendicular to the machine direction.

“Dimension”, “Length”, “Width”, “Pitch”, “Interval”, “Diameter”, “Aspect Ratio”, “Angle”, and “Area” of the article are all measured in a state wherein the article is extended to the Full Circumference according to the “Whole Article Force Measurement” herein, and utilizing a ruler or a loupe, unless specified otherwise.

“Artwork” refers to a visual presentation to the naked eye, which is provided by printing or otherwise, and having a color. Printing includes various methods and apparatus well known to those skilled in the art such as lithographic, screen printing, flexographic, and gravure ink jet printing techniques.

“Color” or “Colored” as referred to herein includes any primary color except color white, i.e., black, red, blue, violet, orange, yellow, green, and indigo as well as any declination thereof or mixture thereof. The color white is defined as those colors having a L* value of at least 94, an a* value equal to 0±2, and a b* value equal to 0±2 according to the CIE L* a* b* color system.

### DETAILED DESCRIPTION

### Wearable Article and Elastic Cover

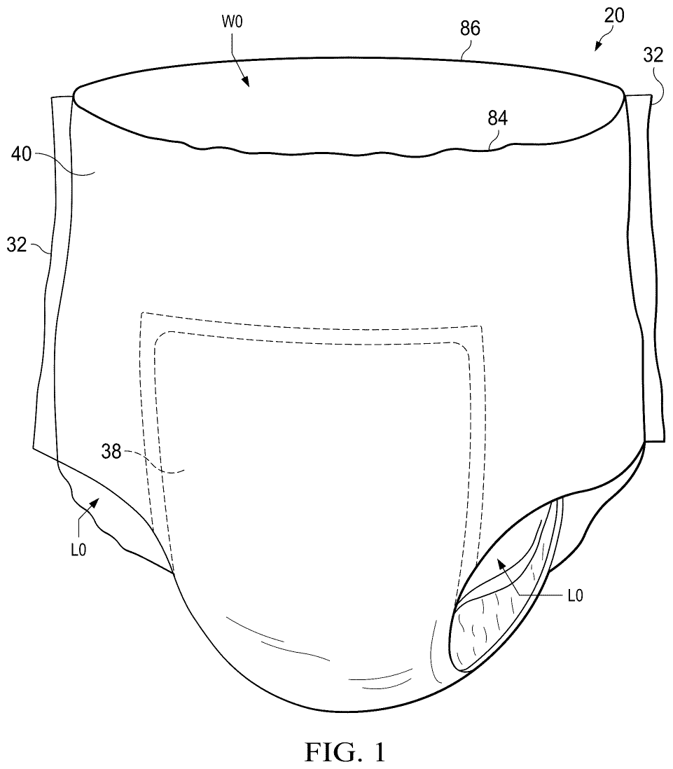

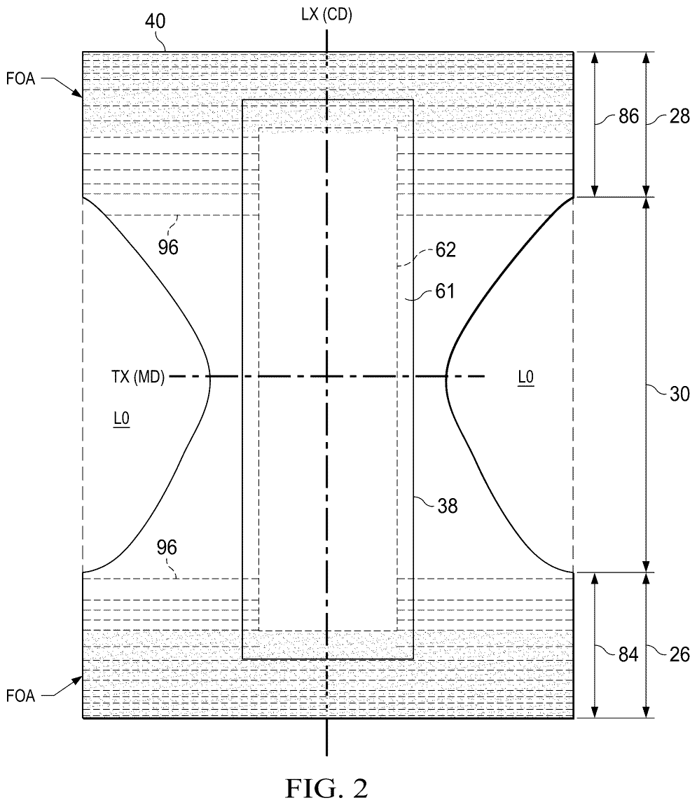

FIG. 1 is a wearable article (20) of the present invention comprising an elastic cover (40) forming the garment facing surface and a center chassis (38) disposed on the wearer facing surface of the elastic cover (40). The wearable article (20) has a waist opening (WO) and two leg openings (LO). FIG. 2 is a schematic plan view of an embodiment of the wearable article (20) of the present invention with the seams (32) unjoined and in its flat uncontracted condition showing the wearer-facing surface. The wearable article (20) as well as the clastic cover (40) has a longitudinal centerline LX which also serves as the longitudinal axis, and a transverse centerline TX which also serves as the transverse axis. The wearable article (20) and the clastic cover (40) has a front region (26) and a back region (28) extending transversely between the side seams (32), and a crotch region (30) positioned longitudinally between the front and back regions (26, 28). The front and back regions of the elastic cover (40) may also be referred to as the front belt region (84) and the back belt region (86). The elastic cover (40) of the present invention acts to dynamically create fitment forces and to distribute the forces dynamically generated during wear. The elastic cover (40) may be made by an inner sheet (92), an outer sheet (92), and a plurality of elastic bodies.

Elastic bodies which extend transversely are defined waist clastic bodies (96) herein. The front belt region (84) and the back belt region (86) may be configured to impart elasticity in the transverse direction to the elastic cover (40) by a plurality of waist elastic bodies (96) running in the transverse direction. At least some of the waist clastic bodies (96) may extend in the transverse direction substantially parallel to each other. All of the waist elastic bodies (96) may extend in the transverse direction substantially parallel to each other.

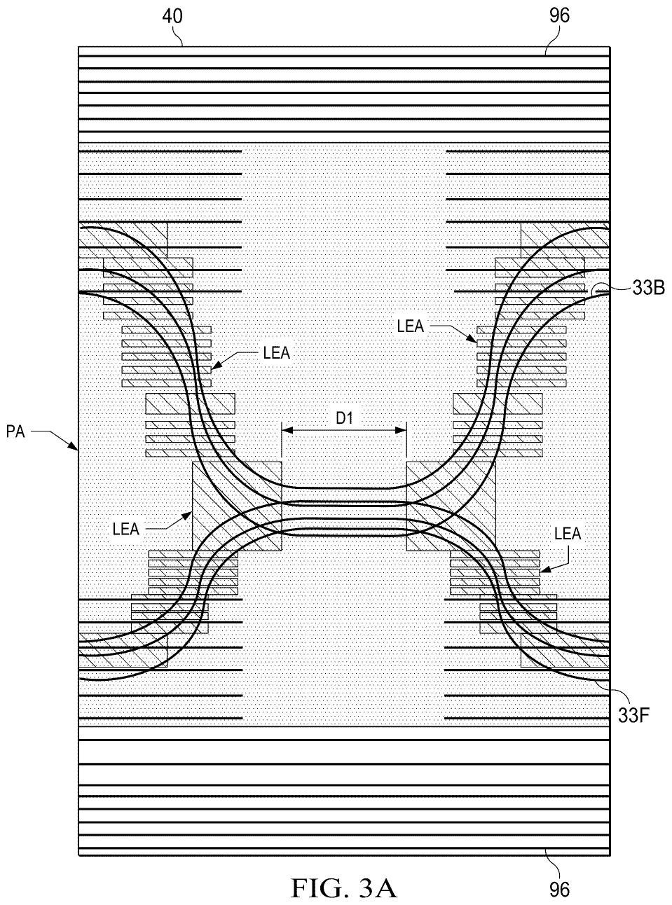

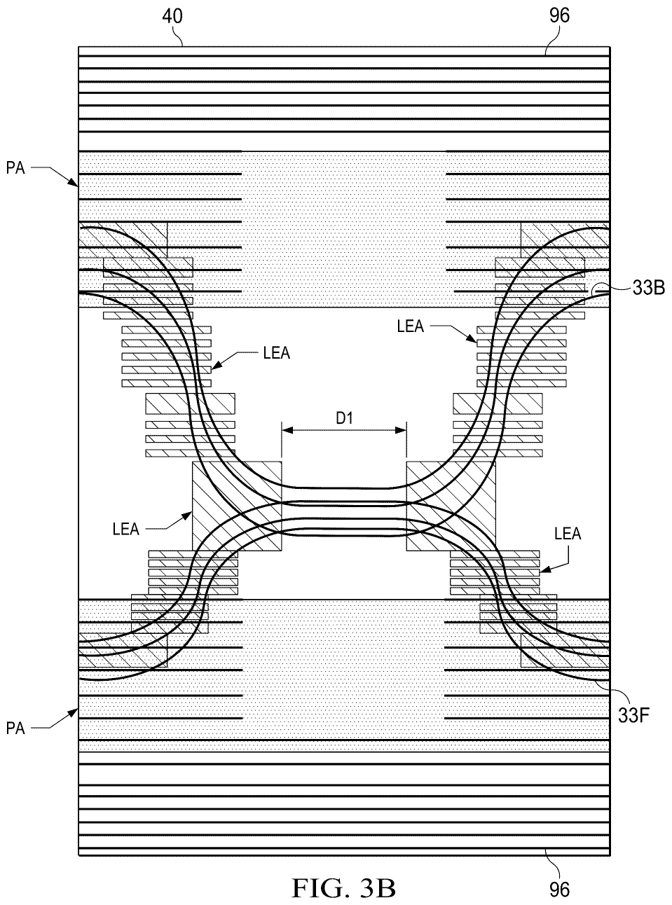

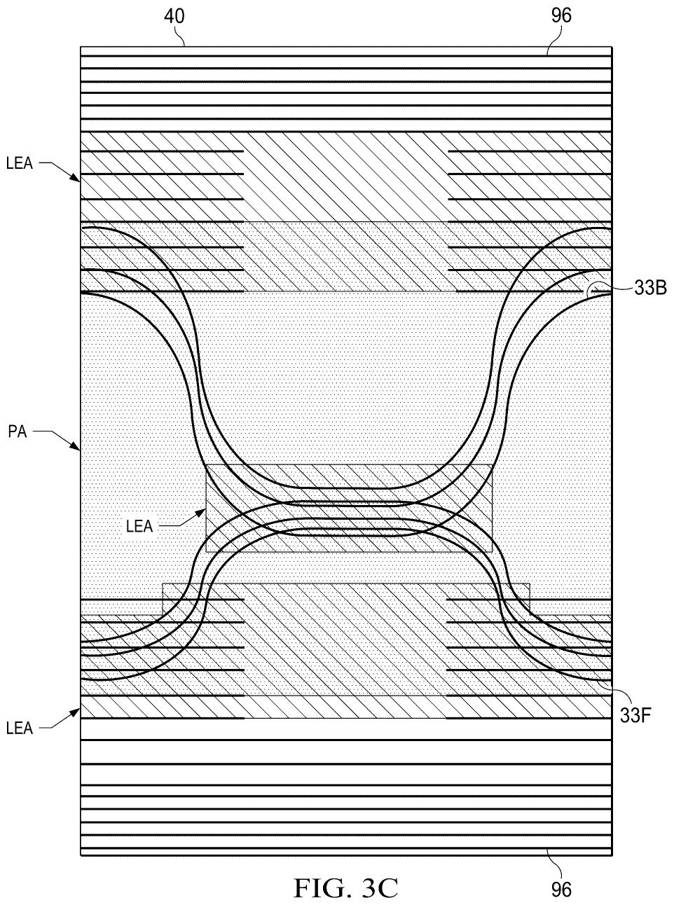

FIGS. 3A-3C are schematic plan views of embodiments of an elastic cover (40) assembly, showing the individual article at a process step of applying the leg elastic bodies on the inner or outer sheet prior to attaching the other of the inner or outer sheet on the assembly. Thereby, the left and right leg openings (LO) are yet to be trimmed off.

Referring to FIGS. 3A-3B, clastic bodies which extend into the crotch region (30) generally along the leg openings (LO) are defined leg clastic bodies (33) herein. A plurality of leg elastic bodies (33) may extend from the proximal edges of the front and back belt regions (84, 86) extending into the crotch region (30) to impart elasticity along the leg opening (LO). The leg clastic bodies (33) may comprise a plurality of front leg elastic bodies (33F) joined between the outer sheet and the inner sheet and extending from the left front region through the crotch region (30) to the right front region, and a plurality of back leg elastic bodies (33B) joined between the outer sheet and the inner sheet and extending from the left back region through the crotch region (30) to the right back region.

The elastic cover (40) may be closely associated with the function and quality of the article (20). Thus, materials for forming the elastic cover (40), as well as the elastic profile of the elastic cover (40), are carefully selected by the manufacturer for providing the desirables for the article (20). An undergarment kind of appearance and pleasant tactile sense such as flexibility and cushiony touch may be associated with high quality. Ease of stretch and application, while also maintaining certain force during wear to prevent the article (20) from sagging after loading, may be associated with high function. The balance of the aforementioned quality and function attributes provide the favorable entire usage experience of the article (20) by the user. While not shown, there may be provided an outer sheet fold over wherein the outer sheet fold over is an extension of the outer sheet (92) material formed by folding the outer sheet (92) material at the distal edge of the front and back belt regions (84, 86) as in FIG. 2. The folding of the outer sheet may be provided after placing the center chassis on the wearer facing sides of the elastic cover (40) and providing the outer sheet fold over, such that the front and back longitudinal edges of the center chassis are covered by the outer sheet fold over.

The waist elastic bodies (96) and leg elastic bodies (33) may be disposed in the same or different denier, interval, and elongation. The waist elastic bodies (96) may be disposed in the same or different force between the front and back, as well as in different longitudinal positions of the elastic cover (40).

Referring back to FIG. 2, the center chassis (38) may comprise a topsheet, a backsheet and an absorbent core (62) disposed between the topsheet and the backsheet. The topsheet may be a water permeable substrate. The backsheet may be a water impermeable film. The center chassis (38) may contain an absorbent core (62) for absorbing and containing body exudates disposed on the center chassis (38), and an absorbent material non-existing region (61) surrounding the periphery of the absorbent core (62). The absorbent material non-existing region (61) may be made of the topsheet and/or the backsheet and/or other parts configuring the center chassis (38). Although not shown, the center chassis (38) may further comprise inner cuffs along the transverse side edges extending in the longitudinal direction.

The front and/or back belt region (84, 86) may be treated such that certain of the area of waist clastic bodies (96) overlapping the center chassis (38) is removed of elasticity. Removal of elasticity from a certain length of the waist elastic bodies (96) disposed on the front and/or back belt region (84, 86) may be advantageous when the center chassis (38) comprises an absorbent core (62), in that elasticity in the front and/or back area overlapping the absorbent core (62) may cause bunching of the absorbent layer or any of the layers in the absorbent core (62) and interfere with close fit of the center chassis (38) to the wearer. The entire area where the elastic bodies overlap with the absorbent core (62) may be removed of its elasticity. By removal of elasticity, what is meant is that the particular portion of the elastic body is non-contributory to elasticity of the elastic cover (40).

### Leg Elastic Body

As mentioned above and referring to FIGS. 3A-3B, the leg elastic bodies (33) may comprise a plurality of front leg elastic bodies (33F) and plurality of back leg elastic bodies (33B). The plurality of front leg elastic bodies (33F) are joined between the outer sheet and the inner sheet and extending from the left front region along the leg opening and, at a position approximately in the longitudinal center of the article, extend transversely through the crotch region (30) to the right front region. The plurality of back leg elastic bodies (33B) are joined between the outer sheet and the inner sheet and extending from the left back region along the leg opening and, at a position approximately in the longitudinal center of the article, extend transversely through the crotch region (30) to the right back region. There may be from about 2 to about 5 each of front leg elastic bodies (33F) and back leg elastic bodies (33B) disposed on the elastic cover (40). Each leg elastic body (33) may be provided in a density of from about 300 dtex to about 1000 dtex, and disposed at an elongation of from about 120% to about 400%. The leg clastic bodies (33) may all be provided in the same dtex and elongation, or have an increase of dtex and/or elongation toward the inward disposed clastic body. The leg elastic bodies (33) may be introduced in the elastic cover (40) such that at any region where the leg elastic bodies (33) are disposed, the leg elastic bodies have a spacing of at least about 3 mm between each other.

The front and back leg elastic bodies (33F, 33B) are joined to the outer sheet or inner sheet by a leg elastic adhesive (LEA) which is a hot melt adhesive applied in slot-die coating. By slot-die coating, what is meant is a coating for producing a thin film of hot melt adhesive by delivering the hot melt adhesive in flowable condition onto the surface of the substrate through a slot-die. The leg elastic adhesive (LEA) may be applied in a density of at least about 3 gsm, or at least about 4 gsm, or at least about 5 gsm, or from about 5 gsm to about 12 gsm.

Still referring to FIGS. 3A-3B, the leg elastic adhesive (LEA) of the present invention is provided in a plurality of stripes intermittent in the longitudinal direction for joining the leg elastic bodies (33), wherein each leg elastic adhesive (LEA) stripe has a longitudinal dimension of no more than about 15 mm, preferably from about 5 mm to about 10 mm, and spaced apart from each other in the longitudinal direction with a spacing of no more than about 25 mm, or a spacing of from about 5 mm to about 15 mm. The leg clastic adhesive (LEA) stripes may each have a transverse dimension of from about 15 mm to about 75 mm, or from about 25 mm to about 65 mm. The transverse dimension of the leg elastic adhesive (LEA) stripes may be consistent. By joining the leg clastic bodies (33) in this manner, a relatively high density of adhesive is provided for attaching the leg elastic bodies (33) to the inner or outer sheet while controlling the total amount of leg clastic adhesive (LEA) applied on the clastic cover (40). A relatively high density of adhesive is advantageous for securely joining the leg elastic bodies (33) to the inner or outer sheet, and preventing the joined leg elastic bodies (33) to be dispositioned from the intended position after loading of the absorbent core during use.

In areas overlapping and not overlapping the regions where the leg elastic adhesive (LEA) is applied, the outer sheet and the inner sheet may be joined with each other by a panel adhesive (PA) which is a hot melt adhesive applied in spiral spray coating. By spiral spray coating, what is meant is a coating provided in a limited area by delivering the hot melt adhesive in flowable condition onto the surface of the substrate through a spray nozzle. The spray nozzle may be in oscillating or circular movement, thereby, for example leaving a trace of spirals. The panel adhesive (PA) may be applied in a density of less than about 2 gsm, or from about 0.5 gsm to about 2 gsm. By joining the inner and outer sheets in this manner, a relatively low density of adhesive is provided for attaching a greater area of the inner or outer sheets, thus controlling the total amount of leg elastic adhesive (LEA) and panel adhesive (PA) applied on the elastic cover (40).

Comparison is made against FIG. 3C of the prior art. In FIG. 3C, leg elastic adhesive (LEA) is provided along the front and back edges of the front and back leg elastic bodies (33) and extending to the distal side of the front and back belt regions, as well as the longitudinal and transverse center of the elastic cover (40). In regions where the leg elastic adhesive is devoid, panel adhesive (PA) is applied for joining the remainder of the crotch region (30). In this embodiment of FIG. 3C, there is a large amount of relatively high density leg elastic adhesive (LEA) applied to the clastic cover (40), thereby making this region stiff and uncomfortable. Yet, in that a relatively large dimension of the back leg elastic bodies (33) are joined to the inner or outer sheet by a relatively low density panel adhesive (PA), the leg elastics in this region risk dispositioning from the original position after loading of the absorbent core, due to pressure exerted against the elastic cover (40) by the bulged absorbent core. Specifically, after loading of the absorbent core, the plurality of clastic bodies (33) positioned adjacent each other may bunch up and behave as a single clastic body of accumulated density. Further, the originally intended clastic distribution, in combination with other clastic bodies of the article for supporting the absorbent core, may be disrupted. Accordingly, dispositioning of plurality of leg elastic bodies (33) leads to higher tendency of causing red marking against the wearer as well as sagging of the article, thus is disadvantageous. In contrast, in FIG. 3A, while there is panel adhesive (PA) applied over a greater region of the elastic cover (40), the area to which relatively high density leg clastic adhesive (LEA) is applied is significantly decreased. Specifically, each leg clastic body (33) may be joined by the leg elastic adhesive (LEA) for from about 20% to about 80%, preferably from about 40% to about 80%, of the length of each leg elastic body. Thus, the overall clastic cover (40) is provided less stiff than that of FIG. 3C, while the leg elastic bodies (33) are securely joined to the inner or outer sheet along the entire dimension of the leg clastic bodies (33). FIG. 3B is an embodiment enabling even further reduction of panel adhesive (PA) in the crotch region (30), providing even less adhesive in this area. The panel adhesive (PA) may be devoid on the leg clastic bodies (33) for at least about 20%, preferably from about 40% to about 70% of the length of each leg elastic body (33).

Referring to FIGS. 3A-3B, the leg elastic adhesive (LEA) may be devoid on the longitudinal axis. By providing the transverse center of the crotch region (30) devoid of leg clastic adhesive (LEA), this maintains the softness of the crotch region (30), and further enables treating the transverse center of the crotch region (30), such that certain of the length of the leg clastic bodies (33) overlapping the center chassis (38) may be removed of elasticity. Similar to the waist elastic bodies (96), removal of elasticity in this region may be advantageous when the center chassis (38) comprises an absorbent core (62) for preventing bunching and wrinkling of the absorbent layer or any of the layers in the absorbent core (62) and thus facilitate good fit and good shape maintenance of the overall article. The leg elastic adhesive stripes (LEA) disposed closest to each other in the transverse direction may have a transverse distance (D1) of from about 30 mm to about 110 mm, preferably from about 50 mm to about 90 mm. The leg elastic bodies (33) positioned in the transverse center of the crotch region (30) may be applied with the lower gsm panel adhesive (PA) and devoid of the higher gsm leg elastic adhesive (LEA), in order to cut and deactivate the leg clastic bodies (33) in this area. In order to further facilitate cutting of the leg clastic bodies (33) in this area, the front and back leg elastic bodies (33F, 33B) may overlap in this area.

### Process

Referring to FIGS. 2 and 3A-3B, the elastic cover (40) of the present invention may be conveniently made by a method of manufacture wherein the machine direction (MD) matches the transverse direction (TX) of the obtained elastic cover, the cross machine direction (CD) matches the longitudinal direction (LX) of the obtained elastic cover, and wherein the assembly for providing individual elastic covers (40) are continuous in the machine direction. The elastic cover (40) of the present invention may be conveniently made by a method of manufacture comprising the steps of:
advancing two layers of continuous sheets in a machine direction;
advancing a plurality of first elastic bodies in the machine direction;
advancing a plurality of second elastic bodies in the machine direction;
stretching the first elastic bodies in the machine direction which oscillate in the cross machine direction;
stretching the second elastic bodies in the machine direction which oscillate in the cross machine direction;
intermittently adhering the first and second elastic bodies between the two layers by a leg elastic adhesive which is a hot melt adhesive applied in slot-die coating; wherein the leg elastic adhesive is provided in a plurality of stripes intermittent in the cross machine direction for joining the first and second elastic bodies, wherein each leg elastic adhesive stripe has a cross machine dimension of no more than about 15 mm, and spaced apart from each other in the cross machine direction with a spacing of no more than about 25 mm;
trimming out a plurality of elastic leg holes; and
cutting the obtained continuous assembly into individual elastic covers (40).

The two layers of continuous sheets result in the inner and outer sheets. The first and second elastic bodies result in the front and back leg elastic bodies (33).

The method may further comprise the step of cutting the first and second leg elastics in a position where the leg elastic adhesive is not applied.

The method may have the two layers of continuous sheets joined by a panel adhesive (PA) which is a hot melt adhesive applied in spiral spray coating. As in FIG. 3B, the method may have the panel adhesive (PA) devoid in the cross machine center.

The leg elastic bodies (33) may be disposed on the inner and outer sheet immediately before or after disposing the waist elastic bodies (96) on the sheets.

Referring to FIG. 2, prior to the step of cutting the obtained continuous assembly into individual elastic covers (40), the center chassis (38) may be placed on the planned-to-be wearer facing side of the continuous elastic cover (40) assembly, followed by applying a fold over adhesive (FOA) to the waist opening edges of the assembly, followed by providing an outer sheet fold over by folding an extended portion of the outer sheet.

### EXAMPLES

Examples 1-2 and Comparative Example 1 are Size 4 (L-size) pant diaper articles having waist elastic bodies (96), leg elastic bodies (33), as well as leg elastic/panel adhesive (LEA/PA) in the configurations of FIGS. 3A-3C respectively, and formed into individual articles. In FIGS. 3A and 3B, the leg elastic adhesive (LEA) is provided in stripes planning to have a longitudinal dimension of from 5 mm to 15 mm and spaced apart from each other with a spacing of 5 mm. The amount and type of adhesive planned for manufacturing each example is provided in Table 1 below.

TABLE 1

| | Exam- | Exam- | Comparative |
|---|---|---|---|
| Planned Adhesive Density/Amount | ple 1 | ple 2 | Example 1 |
| Adhesive configuration | FIG. 3A | FIG. 3B | FIG. 3C |
| Leg elastic adhesive (gsm) | 8 | 8 | 1.9 |
| Panel adhesive (gsm) | 1 | 1 | 2.7 |
| Leg elastic adhesive (grams per article) | 0.206 | 0.206 | 0.147 |
| Panel adhesive (grams per article) | 0.171 | 0.065 | 0.360 |
| Total adhesive (grams per article) | 0.377 | 0.271 | 0.507 |

Examples 1, 2 and Comparative Example 1 may be made at the same production speed. Comparative Example 1, compared to Example 1 or 2, has a higher risk of the leg elastics being dispositioning from the original position after loading of the absorbent core, due to lower density and amount of leg elastic adhesive provided.

Samples of Example 1 and Comparative Example 1 were subject to a consumer acceptance test as follows.

### Consumer Acceptance Test

Example 1 and Comparative Example 1 having identical configurations except for the leg elastic adhesive and panel adhesive were subjected to a consumer test for application on 120 panelists, respectively. The panelists were caregivers of Chinese Size 4 (L-size) wearers of age 0-36 months, and at about the same boy/girl ratio. The panelists were given enough samples of both Example 1 and Comparative Example 1 to use each sample for 5 days. Half of the panelists used Example 1 first and then Comparative Example 1, while the other half of the panelists used Comparative Example 1 first and then Example 1. After the total of 10 days usage, all the panelists answered a questionnaire including softness assessment in which they were asked to rate the performance in 5 scales from “Very Poor” to “Excellent”, wherein 100 represents “Excellent”, 75 represents “Good”, 50 represent “Fair”, 25 represent “Poor” and 0 represents “Very Poor”. The ratings were averaged and statistically analyzed. As a result, the softness rating of the sample of Example 1 was statistically significantly higher than that of Comparative Example 1, as shown in Table 2 below.

TABLE 2

| | Example 1 | Comparative Example 1 |
|---|---|---|
| Softness rating | 78 | 71 |

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.” Further, every numerical range given throughout this specification includes every narrower numerical range that falls within such broader numerical range.

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An elastic cover having a longitudinal direction, a transverse direction, a pair of side seams, and a pair of leg openings, wherein the elastic cover is formed by joining the pair of side seams comprising:
a front region transversely extending between the pair of side seams;
a back region transversely extending between the pair of side seams; and
a crotch region longitudinally extending between the front and back regions;
the elastic cover comprising an elastic laminate comprising:
an outer sheet,
an inner sheet,
a plurality of waist elastic bodies joined between the outer sheet and the inner sheet and extending transversely substantially parallel to each other, and
a plurality of front leg elastic bodies joined between the outer sheet and the inner sheet and extending from the left front region through the crotch region to the right front region,
a plurality of back leg elastic bodies joined between the outer sheet and the inner sheet and extending from the left back region through the crotch region to the right back region,
wherein the front and back leg elastic bodies are joined to the outer sheet or inner sheet by a leg elastic adhesive which is a hot melt adhesive applied in slot-die coating;
wherein the leg elastic adhesive is provided in a plurality of stripes intermittent in the longitudinal direction for joining the leg elastic bodies, wherein each leg elastic adhesive stripe has a longitudinal dimension of no more than about 15 mm, and spaced apart from each other in the longitudinal direction with a spacing of no more than about 25 mm.

2. The elastic cover of claim 1, wherein the leg elastic adhesive is applied in a density of at least about 3 gsm.

3. The elastic cover of claim 1, wherein each leg elastic body is joined by the leg elastic adhesive for no more than about 80% of the length of each leg elastic body.

4. The elastic cover of claim 1, wherein the leg elastic bodies are provided in a density of from about 300 dtex to about 1000 dtex, and an elongation of from about 120% to about 400%.

5. The elastic cover of claim 1, wherein the outer sheet and the inner sheet are joined with each other by a panel adhesive which is a hot melt adhesive applied in spiral spray coating in a density of less than about 2 gsm.

6. The elastic cover of claim 5, wherein the panel adhesive is applied on the leg elastic bodies.

7. The elastic cover of claim 6, wherein the panel adhesive is devoid on the leg elastic bodies for at least 20% of the length of each leg elastic body.

8. A method of manufacturing an elastic cover comprising steps of:
advancing two layers of continuous sheets in a machine direction;
advancing a plurality of first elastic bodies in the machine direction;
advancing a plurality of second elastic bodies in the machine direction;
stretching the first elastic bodies in the machine direction which oscillate in the cross machine direction;
stretching the second elastic bodies in the machine direction which oscillate in the cross machine direction;
intermittently adhering the first and second elastic bodies between the two layers by a leg elastic adhesive which is a hot melt adhesive applied in slot-die coating; wherein the leg elastic adhesive is provided in a plurality of stripes intermittent in the cross machine direction for joining the first and second elastic bodies, wherein each leg elastic adhesive stripe has a cross machine dimension of no more than about 15 mm, and spaced apart from each other in the cross machine direction with a spacing of no more than about 25 mm;
trimming out a plurality of leg openings; and
cutting the obtained continuous assembly into individual elastic covers.

9. The method of claim 8, further comprising a step of cutting the first and second leg elastic bodies in a position where the leg elastic adhesive is not applied.

10. The method of claim 8, wherein the two layers of continuous sheets are joined by a panel adhesive which is a hot melt adhesive applied in spiral spray coating.

11. The method of claim 10, wherein the panel adhesive is devoid in the cross machine center.
